(19) United States
(12) Patent Application Publication
(10) Pub. No.: US 2009/0028951 A1
(43) Pub. Date: Jan. 29, 2009

(54) Compositions for Oral Administration of Sustained Release Glutathione, Methods for Their Production and Uses Thereof

(76) Inventor: Al Czap, Dover, ID (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/995,680

(22) PCT Filed: Jul. 13, 2006
(86) PCT No.: PCT/US06/27126

§ 371 Date: May 15, 2008

Related U.S. Application Data
(60) Provisional application No. 60/698,478, filed on Jul. 13, 2005.

(51) Int. Cl. A61K 38/06; A61K 9/14; A61P 25/28; A61P 21/00; A61P 25/08; A61P 35/00

(52) U.S. Cl. 424/489; 514/18

(57) ABSTRACT

A composition suitable for oral administration for sustained-release of glutathione, the composition having from about 50% by weight to about 90% by weight glutathione; from about 0% by weight to about 10% by weight binder; from about 10% by weight to about 50% by weight of at least one sustained release agent; and a granule size of from about 850 μm to about 5000 μm, is provided. Methods for preparing such compositions are also provided. Uses and methods of medical treatment by orally administering a composition for sustained release of glutathione to a patient suffering from or at risk of suffering from a neurological disorder are also provided.

### BACKGROUND

Glutathione is a tripeptide often referred to as GSH (gamma-glutamyl-cysteinyl-glycine). Glutathione is known to function directly and indirectly in many important biological mechanisms and is also known to be produced by the liver and distributed throughout a body via the bloodstream. Its pharmacological use is well known, for example in perenteral and oral pharmaceutical compositions for the treatment of ethanol intoxication and for the prevention of the toxic effects of chemotherapeutic agents.

Oral drug delivery of proteins and peptides has been described as a challenge because it can be difficult to avoid breakdown of the peptide by the digestive system. In particular, it can be difficult to deliver the protein or peptide past the stomach where pepsins and other enzymes digest proteins. In many cases, the solution has been to protect the peptide with gastroresistant materials to prevent or reduce digestion of the protein or peptide in the stomach.

Prescribing medicine to elderly patients may be particularly difficult for a number of reasons, including the number of drugs being prescribed and the changes in the normal physiological conditions that occur with age. It is known that elderly patients may have a poorer absorption of food and pharmaceuticals due to deterioration of the digestive tract, including a decrease in the production of gastric hydrochloric acid. The decrease in the production of gastric hydrochloric acid often leads to low gastric acidity.

One of the signs of low gastric acidity is the presence of undigested food in the stool. Furthermore, it is known that orally administered medication may also appear in the stool of people with low gastric acidity, often resulting in reduced efficacy of medication.

### SUMMARY

This invention provides methods and compositions for oral administration of glutathione across a range of gastric pHs, including formulations for sustained-release of glutathione. In accordance with one aspect of the invention, compositions comprising glutathione suitable for oral administration to a patient suffering from low gastric acidity may be prepared by controlling the granule size of the composition.

In one aspect, there is provided a method of medical treatment comprising orally administering a composition for sustained release of glutathione to a patient in need thereof. In another aspect, there is provided a use of a composition suitable for oral administration for sustained release of glutathione for treatment of a patient. in another aspect, there is provided a use of a composition suitable for oral administration for sustained release of glutathione for preparation of a medicament for treatment of a patient. The patient in such cases may for example be suffering from Alzheimer's disease, Parkinson's disease or Autism.

In another aspect, there is provided a method for preparing a sustained-release composition containing glutathione, suitable for oral administration, the method comprising: a) mixing glutathione and a binder, thereby forming a mixture; b) adding a granulating agent to the mixture, thereby forming a wet mixture; c) granulating the wet mixture, thereby forming a granulation, such that the granulation comprises granules larger than about 850 μm; d) removing granules less than 850 μm from the granulation; e) blending at least one sustained release agent with the granulation, thereby forming a blend; f) screening the blend with an 850 μm mesh, thereby forming a screened blend; and g) reblending the screened blend. The method may further comprise mixing a glidant or leucine with the composition. The method may further comprise encapsulating the composition. The mixing may occur for a duration of from about 2 minutes to about 6 minutes or about 4 minutes. The adding may occur for a duration of from about 4 minutes to about 9 minutes or about 6.5 minutes. The blending may occur for a duration of from about 2 minutes to about 6 minutes or about 4 minutes. The reblending may occur for a duration of from about 2 minutes to about 6 minutes or about 4 minutes.

In another aspect, there is provided a composition suitable for oral administration for sustained-release of glutathione, the composition comprising: a) from about 50% by weight to about 90% by weight glutathione; b) from about 1% by weight to about 10% by weight binder; c) from about 10% by weight to about 50% by weight of at least one sustained release agent; and d) a granule size of from about 850 μm to about 5000 μm. The composition may provide sustained release of glutathione over a period of from about 2 hours to about 12 hours or from about 4 hours to about 8 hours. The granule size may be from about 500 μm to about 2000 μm, about 500 μm to about 1000 μm, about 500 μm to about 850 μm, about 750 μm to about 850 μm or about 850 μm. The composition may further comprise a glidant. The glidant may be silicon dioxide. The composition may further comprise leucine. The glutathione may be present at from about 60% by weight to about 80% by weight or at about 70% by weight. The binder may be present at from about 1% by weight to about 5% by weight or at about 2.8% by weight. The at least one sustained release agent is present at from about 20% by weight to about 40% by weight or at about 27.2% by weight. The binder may be Kollidon™ 30. The at least one sustained release agent may be Methocel™ KM100P CR and/or Methocel™ E4M CR. The Methocel™ KM100P CR may be present at about 21.7% by weight and the Methocel™ E4M CR may be present at about 5.5% by weight. The composition may be suitable for administration to a patient having a gastric pH of from about 1 to about 7, from about 2 to about 6, from about 3 to about 5, about 1, about 1.5, about 2, about 2.5, about 3, about 3.5, about 4, about 4.5, about 5, about 5.5, about 6 or about 6.5. A composition may comprise 70% by weight glutathione, 2.8% by weight Kollidon™ 30, 21.7% by weight Methocel™ KM100P CR, 5.5% by weight Methocel™ E4M CR.

In another aspect, there is provided a method of maintaining a serum glutathione concentration in a patient for a duration of from about 2 hours to about 12 hours comprising administering a composition described herein.

In another aspect, there is provided a method of medical treatment comprising administering a composition described herein to a patient suffering from at least one of the diseases selected from the group consisting of: Addison's disease, asthma, Autism, Celiac disease, dermatitis herpetiformis, diabetes mellitus, eczema, gallbladder disease, Graves disease, chronic auto-immune disorders, hepatitis, chronic hives, lupus erythematosis, myasthenia gravis, osteoporosis, pernicious anemia, psoriasis, rheumatoid arthritis, rosacea, Sjorgren's syndrome, thyrotoxicosis, hyperthyroidism, hypothyroidism, vitiligo, Alzheimer's disease, Parkinson's disease, vascular disease, atherosclerosis and asthma. The patient may be suffering from a dementia, Alzheimer's disease or Parkinson's disease. The patient may be further suffering from low gastric acidity.

In another aspect, there is provided a use of a composition described herein for maintaining a serum glutathione concentration in a patient for a duration of from about 2 hours to about 12 hours.

In another aspect, there is provided a use of a composition described herein for treatment of: Addison's disease, asthma, Autism, Celiac disease, dermatitis herpetiformis, diabetes mellitus, eczema, gallbladder disease, Graves disease, chronic auto-immune disorders, hepatitis, chronic hives, lupus erythematosis, myasthenia gravis, osteoporosis, pernicious anemia, psoriasis, rheumatoid arthritis, rosacea, Sjorgren's syndrome, thyrotoxicosis, hyperthyroidism, hypothyroidism, vitiligo, Alzheimer's disease, Parkinson's disease, vascular disease, atherosclerosis and asthma.

In another aspect, there is provided a use of a composition described herein for preparation of a medicament for maintaining a serum glutathione concentration in a patient for a duration of from about 2 hours to about 12 hours.

In another aspect, there is provided a use of a composition described herein for preparation of a medicament for treatment of: Addison's disease, asthma, Autism, Celiac disease, dermatitis herpetiformis, diabetes mellitus, eczema, gallbladder disease, Graves disease, chronic auto-immune disorders, hepatitis, chronic hives, lupus erythematosis, myasthenia gravis, osteoporosis, pernicious anemia, psoriasis, rheumatoid arthritis, rosacea, Sjorgren's syndrome, thyrotoxicosis, hyperthyroidism, hypothyroidism, vitiligo, Alzheimer's disease, Parkinson's disease, vascular disease, atherosclerosis and asthma.

### DETAILED DESCRIPTION

Provided are methods for treating or preventing Alzheimer's disease in a patient by administering glutathione to the patient. In one embodiment, the glutathione is a sustained release composition of glutathione. The present invention is based, in part, on the discovery that glutathione and sustained release glutathione can be orally administered to a patient with any gastric pH, including low gastric acidity.

Further provided are methods for the treatment and prevention of at least one of the following diseases and disorders by administering to a patient a sustained release composition of glutathione: Addison's disease, asthma, Autism, Celiac disease, dermatitis herpetiformis, diabetes mellitus, eczema, gallbladder disease, Graves disease, chronic auto-immune disorders, hepatitis, chronic hives, lupus erythematosis, myasthenia gravis, osteoporosis, pernicious anemia, psoriasis, rheumatoid arthritis, rosacea, Sjorgren's syndrome, thyrotoxicosis, hyperthyroidism, hypothyroidism, vitiligo, Alzheimer's disease, Parkinson's disease, vascular disease, atherosclerosis and asthma. In yet another aspect, a sustained release composition of glutathione may serve as a prophylactic of them or any other disease.

Also provided is a sustained release composition of glutathione and methods of manufacture that render a composition with an optimal release profile. Furthermore, the composition and methods of manufacture render a composition that is conveniently compressible, but not excessively friable.

In another embodiment, the compositions may comprise at least one sustained release agent. In another embodiment, the glutathione may be slowly released into the system of a patient. The slow release of glutathione creates a pharmacokinetic profile of glutathione within the plasma that provides a substantially constant supply of glutathione. The compositions may, therefore, slowly dissolve in vivo and release a substantially uniform amount of glutathione over a time period to be therapeutically effective for a patient.

As used herein, unless otherwise specified, the term “patient” includes mammals. The term “mammals” includes, but is not limited to, dogs, cats, cattle, horses, pigs, and humans.

As used herein, the terms “treat”, “treating”, “treatment” and the like refer to the application or administration of a therapeutic agent or composition to a patient, or application or administration of a therapeutic agent or composition to an isolated tissue from a patient, who has a disease or disorder, a symptom of disease or disorder or a predisposition toward a disease or disorder, with the purpose of curing, healing, alleviating, relieving, altering, remedying, preventing, ameliorating, delaying onset of the disease or disorder and/or event, slowing the progression of the disease or disorder, improving or affecting the disease or disorder, the symptoms of disease or disorder or the predisposition toward a disease or disorder and/or event.

As used herein the terms “coadministration” or “coadministered” when used to describe the administration of two or more compounds to a patient means that the compounds, which may be administered by the same or different routes, are administered concurrently (e.g., as a mixture) or sequentially, such that the pharmacological effects of each overlap in time. As used herein, unless otherwise specified, when applied to the administration of at least two compounds, the term “sequentially” means that the compounds are administered such that the pharmacological effects of each overlap in time. In certain embodiments, agents are coadministered substantially simultaneously. By “substantially simultaneously,” it is meant that the composition of the invention is administered to the patient close enough in time with the administration of at least one additional agent, whereby the agents may exert an additive or even synergistic effect.

The term “carrier” refers to diluents, excipients and the like for use in preparing admixtures of a pharmaceutical composition.

As used herein, the term “dosage form” means a pharmaceutical composition that contains an appropriate amount of active ingredient for administration to a patient, e.g., either in single or multiple doses.

The unit “mg/Kg” as used herein means the mg of agent per Kg of patient body weight.

As used herein, unless otherwise indicated, the term “half-life” means the time taken to decrease the concentration of drug in the blood plasma of the organism by about one half from the drug concentration at the time of administration.

As used herein, unless otherwise specified, the term “immediate release” means that no extrinsic factors delay the in vitro release of one or more drugs.

As used herein, the terms “pharmaceutical composition” or “pharmaceutical formulation,” used interchangeably herein, mean a composition that comprises pharmaceutically acceptable constituents.

As used herein, the term “pharmaceutically acceptable” means the type of composition that would be reviewed and possibly approved by a regulatory agency of the Federal or a state government or listed in the U.S. Pharmacopoeia or other generally recognized pharmacopoeia for use in animals, and more particularly in humans.

As used herein, unless otherwise specified, the term “sustained release” is defined as a prolonged release pattern of one or more drugs, such that the drugs are released over a period of time. A sustained release composition is a composition with release kinetics which results in measurable serum levels of the drug over a period longer than that obtained following IV injection or by administering an immediate release oral dosage form. A sustained release composition may provide a continued effect to drugs of which biological half lives after administration are short; decreasing side effects of drugs which likely exhibit side effect Cₘₐₓ-dependently; and may improve compliance by decreasing the number of times of administration. For purposes of the present invention, sustained release, slow release, controlled release, extended release, prolonged release, and delayed release are used interchangeably.

A “sustained release agent” is defined as a compound or composition that, when included in a composition with one or more drugs, provides a prolonged release pattern of the one or more drugs, such that the drugs are released over a period of time.

As used herein, the term “percent by weight”, “% by weight”, “weight percent” or “weight %” when used to describe the amount of a component within a composition means the weight of the specified component based upon the weight of all components within the composition.

“Low gastric acidity”, as used herein, is a condition whereby the pH of a patient's stomach is higher than normal. Normal stomach pH is between 1 and 2.3. Consequently, a patient suffering from low gastric pH refers to a patient have a stomach pH of from 2.3 and higher. This condition is common among elderly people, as well as many in the general populace.

“Granule”, as used herein, refers to a particle having a size. Powders, blends, mixtures and other compositions may be comprised of particles and/or granules. The granule size of a composition, meaning the size of the granules making up the composition, may impart particular properties to the composition as a whole. Each individual component or ingredient of a composition may have a granule size. Additionally, blends or mixtures of some or all of the components or ingredients of a composition may have a granule size.

Compositions described herein may be suitable for administration to a patient having any gastric pH, including low gastric pH. The patient may have a gastric pH of from about 1 to about 7, or a pH of 1, 2, 3, 4, 5, 6, or 7.

Compositions may comprise glutathione in a therapeutically effective amount and at least one sustained release agent. The compositions may also include additional ingredients necessary to modify the compositions for administration, preservation and esthetics. In one embodiment, the composition of the present invention also include binders, fillers and lubricants. In another embodiment, the composition comprises a sustained release glutathione formula comprising glutathione, a binder, one or more sustained release agents, a glidant, and a release agent or lubricant. The composition may further comprise fillers and/or compression agents.

Glutathione is commercially available from a number of sources known to the skilled practitioner. USP grade glutathione, for example, may be commercially available from various sources including Sigma-Aldrich (Milwaukee, Wis.).

Glutathione may be present at about 10% to about 90% by weight of a composition. In another embodiment, the glutathione may be present at about 25% to about 75% by weight of the composition. In various embodiments, the glutathione may be present at about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In particular embodiments, the glutathione may be present at about 50%, 51%, 56%, 69% or 70%. All ranges within each of the above ranges are within the scope of the present invention.

Compositions may contain less than about 7 g glutathione, for example, less than about 6 g, about 5 g, about 4 g, about 3 g, about 2 g, about 1 g glutathione, or about 0.1 g glutathione. For example, the composition may contain from about 0.1 g to about 7 g, about 2 g to about 6 g or about 3 g to about 5 g glutathione. For example, ranges of values using a combination of any of the above recited values as upper and/or lower limits are intended to be included. The composition may contain less than about 2 g glutathione.

Use of one or more sustained release agents allows for the slow release of the glutathione over an extended period of time. For example, the sustained release agent may release glutathione at a rate that does not cause concentration peaks or lows that may exacerbate side effects associated with high or low concentrations of glutathione within the bloodstream. Sustained release agents suitable for the compositions used in the methods described herein include hydration agents, e.g., cellulose, that partially hydrate when in contact with an aqueous environment to form a gelatinous barrier that retards dissolution of the agent that the hydration agent is coating. The sustained release agents may form a temporary barrier to water such that water is slowly absorbed into the composition thereby hydrating the composition and subsequently releasing the active ingredient, e.g., glutathione, at a rate substantially slower than a composition without a sustained release agent. The sustained release agents may be present in a granule size so that after incorporation into a capsule or compaction or compression into a tablet, pill, or gelcap water may slowly permeate into the structure.

Sustained release agents may include, but are not limited to, cellulose ether products, polymethylmethacrylate, or polyvinylalcohol. In another embodiment, sustained release agents include celluloses including, but not limited to methylcellulose, hydroxypropyl methylcellulose, hydroxyethylcellulose, or combinations thereof. In another embodiment, the sustained release agents include one or more hydroxypropyl methylcelluloses. Suitable sustained release agents are commercially available from The Dow Chemical Company under the trade designations METHOCEL™ and ETHOCEL™. In another embodiment, the sustained release agent is METHOCEL™ K100 M CR Premium and/or METHOCCEL™ E 4M CR Premium.

A sustained release agent may be present in an amount sufficient to release the active ingredient, e.g., glutathione, over a desired period of time. The sustained release agent may be present in an amount of about 5% to about 40% by weight of the composition. The sustained release agent may be present in an amount of about 5% to about 75% by weight. In yet another embodiment, the sustained release agent is present in an amount of about 15% to about 50% by weight of the composition. In various embodiments, the sustained release agent is present at about 5% to about 40%, for example, about 24% to about 25%, about 27% to about 28%, about 31% to about 32%, and about 35%. In alternative embodiments, the sustained release agent is present at about 40% to about 60%, for example, about 45%. Compositions may comprise at least one sustained release agent from about 5% by weight to about 75% by weight, or 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% or 74% by weight. All ranges within each of the above ranges are within the scope of the present invention.

A composition comprising a sustained release agent may release glutathione over a period of 10 hours. The composition may release glutathione substantially uniformly over a period from about 2 hours to about 12 hours. The composition may release glutathione substantially uniformly over a period of about 4 hours to about 8 hours. The sustained release glutathione composition may release glutathione substantially uniformly over a period of about 12 hours to about 48 hours.

Compositions may release glutathione in a manner to provide a pharmacokinetic profile wherein the half-life (T_{1/2}) and the Tₘₐₓ are sufficient to maintain glutathione at a substantially constant level. In one embodiment, a sustained release composition of the invention releases glutathione such that a steady state of circulating glutathione is achieved and remains constant. In one embodiment, the pharmacokinetic profile is such that T_{1/2} is from about 4 hours to about 12 hours and the Tₘₐₓ is about 4 hours. In yet another embodiment, T_{1/2} is from about 4 hours to about 8 hours and the Tₘₐₓ is about 4 hours. In yet another embodiment, T_{1/2} is from about 6 hours to about 9 hours and the Tₘₐₓ is about 2 hours.

Binders that may be included in the composition comprise those commonly known to the skilled practitioner. Binders include, but are not limited to, sugars, such as lactose, sucrose, glucose, dextrose, and molasses; natural and synthetic gums, such as acacia, guar gum, sodium alginate, extract of Irish moss, panwar gum, ghatti gum; other binders include a mixture of polyethylene oxide and polyethylene glycol, methylcellulose, sodium carboxymethylcellulose, hydroxypropyl cellulose (H PC), hydroxyethyl cellulose, hydroxypropyl methylcellulose, alginic acid, ethyl cellulose, microcrystalline cellulose, carbomer, zein, starch, dextrin, maltodextrin, gelatin, pregelatinized starch, polyvinylpyrrolidone (PVP) or povidone, and mixtures thereof. In another embodiment, the binder may be polyvinylpyrrolidone homopolymer, or the polyvinylpyrrolidone sold under the designation Kollidon™.

A binder may be present at less than about 20% by weight of the composition. The binder may be present at about 0.5% to about 10%, for example, about 0.5% to about 5%, about 2% to about 3%, about 3% to about 4%, about 4% to about 5%, about 5% to about 6%, about 6% to about 7%, about 7% to about 8%, about 8% to about 9%, or about 9% to about 10%. Compositions may comprise a binder from about 0% by weight to about 10% by weight, or about 1%, 2%, 3%, 4%, 5%, 6%, 7%, 8%, or 9% by weight. All ranges within each of the above ranges are within the scope of the present invention.

Compositions of sustained release glutathione may also include a glidant. The glidant may be any known USP grade glidant including, e.g., silicon dioxide. The glidant may be colloidal silicone dioxide.

A glidant may be present at less than about 3% by weight of the composition. In some embodiments, the glidant may be present at less than about 2% of the composition. In another embodiment, the glidant is present at less than about 1% by weight of the composition.

Fillers that may be included in a composition comprise those commonly known to the skilled artisan. Fillers may include, but are not limited to, sugars such as lactose, sucrose, dextrose, mannitol, and sorbitol, whey, dibasic calcium phosphate, tribasic calcium phosphate, calcium sulfate, and mixtures thereof. Other fillers include, but are not limited to, cellulose preparations such as maize starch, wheat starch, rice starch, potato starch, gelatin, gum tragacanth, methyl cellulose, hydroxypropyl methylcellulose, sodium carboxymethylcellulose, polyvinylpyrrolidone, and mixtures thereof. Microcrystalline cellulose can also function as a compression agent as well as a filler. In some embodiments the filler/compression agent may be microcrystalline cellulose and/or the microcrystalline cellulose is that sold under the designation AVICEL™ PH 102 by The Dow Chemical Company.

A filler may be present at less than about 50% by weight of the composition. The filler may be present at about 2% to about 20% by weight of the composition including, for example, at about 8% to about 9%, at about 9% to about 10%, at about 10% to about 11%, at about 11% to about 12%, and at about 12% to about 13% by weight of the composition. The filler may be present at about 10% by weight of the composition. All ranges within each of the above ranges are within the scope of the present invention.

Excipients can be added to increase the amount of solids present in the composition.

Excipients that may be used for this purpose include sodium or potassium phosphates, calcium carbonate, calcium phosphate, sodium chloride, citric acid, tartaric acid, gelatin, and carbohydrates such as dextrose, sucrose, lactose, sorbitol, inositol, mannitol and dextran, starches, cellulose derivatives, gelatin, and polymers such as polyethylene glycols. In addition to those mentioned herein, others are known to those skilled in the art. The excipients may also be used in combination with other excipients.

Release agents or lubricants that may be included in the composition comprise those commonly known to the skilled artisan. Lubricants may be chosen so as to insure optimal absorption and utilization of nutrients. Lubricants include, but are not limited to, stearate, magnesium stearate, zinc stearate, calcium stearate, stearic acid, hydrogenated vegetable oils (e.g., hydrogenated cottonseed oil), sodium stearyl fumarate, glyceryl palmitostearate, glyceryl behenate, sodium benzoate, sodium lauryl sulfate, magnesium lauryl sulfate, mineral oil, talc, and mixtures thereof.

A lubricant may be present at less than about 20% by weight of the composition. The lubricant may be present at about 2% to about 20% by weight of the composition. The lubricant may be present at about 10% by weight of the composition.

Disintegrants include, but are not limited to, citric acid alone or in combination with bicarbonate, sodium starch glycolate, croscarmellose sodium, crospovidone, cross-linked polyvinylpyrrolidone, corn starch, pregelatinized starch, microcrystalline cellulose, alginic acid, amberlite ion exchange resins, polyvinylpyrrolidone, polysaccharides, sodium carboxymethylcellulose, agar, salts thereof such as sodium alginate, Primogel, and mixtures thereof.

A compression agent may allow for the composition to be shaped into a tablet, troche, gelcap, or other presentation for administration in solid form. The compression agent may allow the composition to be shaped into a tablet, troche, or gelcap. Compression agents include, but are not limited to, Avicel™, magnesium stearate, wax, gums, celleusics, stearate, or combinations thereof. The compression agent may be microcrystalline cellulose.

A compression agent may be present in an amount of about 0.01% to about 5% by weight percent of the composition. The compression agent may be present in an amount of about 0.5% to about 3%. The compression agent may be present in an amount of about 1% to about 2% by weight of the composition.

Compositions may comprise a pharmaceutical carrier according to conventional pharmaceutical compounding techniques. The carrier may take a wide variety of forms depending on the form of the preparation desired for oral administration. Compositions may be incorporated into a capsule or formed as tablets or gelcaps.

Tablets or capsules may contain a composition described herein in the same tablet or capsule in different configurations. Configurations may include, a two-part half and half tablet or capsule, one composition surrounding a second, dispersion of one composition in another, granules of both compositions intermixed, and the like. If desired, tablets or capsules may be coated by standard aqueous or non-aqueous techniques.

Compositions may also comprise other pharmaceutically acceptable ingredients, such as those commonly used in the art. See, Remington: the Science & Practice of Pharmacy, by Alfonso R. Gennaro, 20th ed., Williams & Wilkins, 2000. Additional ingredients used in the compositions described herein may include, but are not limited to, water, glycols, oils, alcohols, starches, sugars, diluents, disintegrating agents, preservatives, excipients, lubricants, disintegrants, diluents, carriers, stabilizing agents, coloring agents, flavoring agents, and combinations thereof. Examples of suitable diluents include water, ethanol, polyols, vegetable oils, injectable organic esters such as ethyl oleate, and combinations thereof. Compositions may also contain adjuvants such as preserving, wetting, emulsifying, and dispensing agents. Prevention of the action of microorganisms may be achieved by various antibacterial and antifungal agents including, but not limited to, parabens, chlorobutanol, phenol, sorbic acid, and the like. It may also be desirable to include isotonic agents including, but not limited to, sugars, sodium chloride, and the like.

In alternative embodiments, compositions described herein may comprise a granule size of from about 500 μm to about 2000 μm, or 550 μm, 600 μm, 650 μm, 700 μm, 750 μm, 800 μm, 850 μm, 900 μm, 950 μm, 1000 μm, 1050 μm, 1100 μm, 1150 μm, 1200 μm, 1250 μm, 1300 μm, 1350 μm, 1400 μm, 1450 μm, 1500 μm, 1550 μm, 1600 μm, 1650 μm, 1700 μm, 1750 μm, 1800 μm, 1850 μm, 1900 μm or 1950 μm.

In alternative embodiments, compositions may be co-administered with at least one other pharmaceutical agent. Examples of pharmaceutical agents include: adrenergic agent; adrenocortical steroid; adrenocortical suppressant; aldosterone antagonist; amino acid; ammonia detoxicant; anabolic; analeptic; analgesic; androgen; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; anti-infective; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinesterase deactivator; coccidiostat; cognition adjuvant; depressant; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; keratolytic; LNRII agonist; liver disorder treatment; luteolysin; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; potentiator; progestin; prostaglandin; prostate growth inhibitor; prothyrotropin; psychotropic; radioactive agent; regulator; relaxant; repartitioning agent; scabicide; sclerosing agent; sedative; selective adenosine Al antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; treatment of cerebral ischemia; treatment of Paget's disease; treatment of unstable angina; uricosuric; vasoconstrictor; vasodilator; vulnerary; wound healing agent; or xanthine oxidase inhibitor.

Methods for the treatment and prevention of Alzheimer's disease by administering to a patient a composition described herein are also provided. In one embodiment, the composition to be administered is a sustained release composition of glutathione. Furthermore, methods of treating and preventing other indications described herein by administering to a patient a sustained release composition of glutathione are also provided.

Compositions described herein may be used to maintain a serum glutathione concentration in a patient for a duration of from about 2 to 24 hours. In various embodiments, the duration of serum concentration maintenance may be 2 hours, 3 hours, 4, hours, 5 hours, 6 hours, 7 hours, 8 hours, 9 hours, 10 hours, 11 hours, 12 hours, 13 hours, 14 hours, 15 hours 16 hours, 17 hours, 18 hours, 19 hours, 20 hours, 21 hours, 22 hours, 23 hours or 24 hours. This may be achieved by oral administration of the composition.

Compositions described herein may be used for medical treatment of a patient suffering from at least one of the diseases selected from the group consisting of: Addison's disease, asthma, Autism, Celiac disease, dermatitis herpetiformis, diabetes mellitus, eczema, gallbladder disease, Graves disease, chronic auto-immune disorders, hepatitis, chronic hives, lupus erythematosis, myasthenia gravis, osteoporosis, pernicious anemia, psoriasis, rheumatoid arthritis, rosacea, Sjorgren's syndrome, thytotoxicosis, hyperthyroidism, hypothyroidism, vitiligo, Alzheimer's disease, Parkinson's disease, vascular disease, atherosclerosis, asthma and glutathione deficiency. This may be achieved by oral administration of the composition.

Compositions described herein may be used for treating patients in need of treatment with glutathione and having low gastric acidity. This may be achieved by oral administration of the composition.

In other aspects, there is provided methods for preventing or treating Alzheimer's disease by administering glutathione to the patient, a sustained release composition of glutathione, or an immediate release composition of glutathione.

Administration of a composition as a prophylactic agent may occur prior to the manifestation of symptoms characteristic of the onset of the particular indication, such that the disease or disorder is prevented, its progression slowed, or its onset delayed.

The actual amount of compound delivered, as well as the dosing schedule necessary to achieve the desired pharmacokinetic profiles will depend, in part, on such factors as the bioavailability of the compound (and/or an active metabolite thereof), the disorder being treated, the desired therapeutic dose, and other factors that will be apparent to those of skill in the art. The actual amount delivered and dosing schedule can be readily determined by those of skill without undue experimentation by monitoring the blood plasma or serum levels of glutathione and/or an active metabolite thereof, and adjusting the dosage or dosing schedule as necessary to achieve the desired pharmacokinetic profile.

Compositions described herein may be delivered to a patient so as to avoid or reduce undesirable side effects using a wide variety of routes or modes of administration. In one embodiment, the patient may be an animal. In another embodiment, the patient may be a mammal. In yet another embodiment, the patient may be a human. The most suitable route in any given case will depend on the nature and severity of the condition being treated. Often, the route of administration is the oral route.

Compositions may be used in an amount effective to achieve the intended purpose, e.g., to treat or prevent Alzheimer's disease or Parkinson's disease or Autism. A therapeutically effective amount is an amount effective to treat a disease, disorder, symptom related to a disease or disorder, or predisposition toward a disease or disorder. The term “treat” refers to the application or administration of a therapeutic agent or composition to a patient, or application or administration of a therapeutic agent or composition to an isolated tissue from a patient, who has a disease or disorder, a symptom of disease or disorder or a predisposition toward a disease or disorder, with the purpose of curing, healing, alleviating, relieving, altering, remedying, ameliorating, delaying onset of the disease or disorder and/or event, slowing the progression of the disease or disorder, improving or affecting the disease or disorder, the symptoms of disease or disorder or the predisposition toward a disease or disorder and/or event. Determination of a therapeutically effective amount is well within the capabilities of those skilled in that art, especially in light of the detailed disclosure provided herein.

Pharmaceutical compositions include compositions wherein glutathione is contained in a therapeutically effective amount, i.e., an amount effective to achieve the intended purpose. An effective amount may be that amount of a pharmaceutical preparation that alone, or together with further doses, produces the desired response. This may involve only slowing the progression of the disease temporarily. In another embodiment, it involves halting the progression of the disease permanently or delaying the onset of or preventing the disease or condition from occurring. The effect of the dosage on any particular disease can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge of the skilled practitioner.

Doses of active compounds may be from about 0.01 mg/kg per day to about 1000 mg/kg per day. In one embodiment, doses ranging from about 1 to about 50 mg/kg may be suitable. In another embodiment, administration may be oral and in one or several administrations per day.

In another embodiment, the patient may receive less than about 10 g sustained release glutathione per day for example, less than about 9 g, about 8 g, about 7 g, about 6 g, about 5 g, about 4 g, about 3 g, about 2 g, about 1 g, or about 0.1 g sustained release glutathione per day. For example, the patient may receive a daily dosage of from about 0.1 g to about 7 g, about 2 g to about 6 g or about 3 g to about 5 g sustained release glutathione. Ranges of values using a combination of any of the recited values as upper and/or lower limits are intended to be included. The patient may receive less than about 2 g sustained release glutathione per day.

The specific amount of glutathione may depend on, among other things, the condition of the patient, and the weight and metabolism of the patient. Compositions may be tailored to contain an amount of glutathione effective to, inter alia, ameliorate the harmful effects of the particular targeted disease or disorder, i.e., prevent the development of or alleviate the existing symptoms of, or prolong the survival of, the patient being treated. Determination of an effective amount is well within the capabilities of those skilled in the art, and in light of the disclosure herein.

Therapeutically effective amounts for use in humans may also be estimated from animal models. For example, a dose for humans may be formulated to achieve a plasma or serum concentration found to be effective in animals.

Adjusting the dose to achieve maximal efficacy in patients based on the methods known to skilled practitioners. Blood concentration and duration of administered compound and/or its active metabolites is well within the capabilities of the ordinary skilled artisan.

Methods for preparing a composition containing glutathione, suitable for oral administration to a patient having low gastric acidity are provided.

Methods may involve mixing glutathione with a binder. The ratio, by weight, of glutathione to binder to be mixed may be in the range of 1000:1 to 10:1 and may be about 100:1. The glutathione and the binder may be mixed for a duration of 2, 3, 4, 5 or 6 minutes or until the dry blend is homogenous throughout. Various apparatuses for mixing are well known in the art, including the Bohle™ VMA20 and other similar machines.

Once mixed, a solution of the binder may be added to the mixture. The solution may be prepared using a predetermined weight of binder and adding water or purified water to form a solution. The solution may have a concentration range of 800:100 to 200:100 and may be about 633:150. For example, 633 grams of water may be added to 150 grams of binder to prepare a suitable solution.

The solution may be added by pressurized or vacuum assisted nozzle spray, or other appropriate method to a mixture of glutathione and binder over a period of 2, 3, 4, 5, 6, 7, 8, 9 or 10 minutes or until the resulting wet mixture is thoroughly coated and of a consistency which will allow optimal flow in a fluid bed granulator. Various apparatuses and techniques for adding solutions to mixtures are well known in the art, including the Bohle™ VMA20.

A wet mixture may be granulated to form a granulation so that the granulation contains granules that are not less than about 850 μm. Granulation is often achieved by drying the wet mixture using a fluidbed granulator. The fluidbed granulator is often set so that the maximum granule size is achieved during drying.

A granulation may then be screened to remove granules that are too large, or small. A standard U.S. 20 mesh is used to screen the granulation, but mesh sizes ranging from 10 to 30 may be used. The granulation should be screened so that the resulting granulation passes through a standard U.S. 20 mesh.

Mesh sizes correspond to a nominal screen opening in a mesh. For example a standard US 20 mesh has a nominal mesh size of 850 μm. Each mesh has a standard wire thickness, for example, a standard JS 20 mesh has a nominal wire diameter of 0.510 mm. Meshes and their standard characteristics and nomenclature are well understood in the art.

A screened granulation may be blended with at least one sustained release agent. Blending of the screened granulation and the sustained release agent is achieved by adding a predetermined quantity of sustained release agent, though a mesh of predetermined size (e.g. a standard US 20 mesh) a blender containing the screened granulation. Blending may be performed for a duration of 1, 2, 3, 4, 5, 6, 7, or 8 minutes or until the dry blend is homogenous throughout. Various blending techniques and apparatuses are known in the are and often, though not limited to, a V-blender is used for blending. A suitably sized blender may be selected based on the quantity of starting materials.

Once blended, the resulting blend may be dropped through an appropriately sized mesh (e.g. a standard US 20 mesh). This ensures that the blend contains a consistent maximum granule size and that all material passes through the chosen mesh, in this case 20 mesh. After screening, the blend may be added to the blender again and reblended. Reblending may me performed for a duration, of 1, 2, 3, 4, 5, 6, 7 or 8 minutes or until the dry blend is domogenous throughout.

Methods described herein may also include a step whereby other compounds are mixed into the composition. For example, a filler, a glidant, an excipient, a release agent, a lubricant, a disintegrant, a compression agent, a pharmaceutical carrier, another pharmaceutical agent, and other pharmaceutically acceptable ingredients.

Once all of the ingredients of the composition have been added, mixed or blended into the composition, the composition may be encapsulating, compressed into a tablet or added to a food product.

Controlled incorporation and/or coverage of glutathione granules within a matrix may improve the sustained release characteristics of the compositions described herein. In the case of a cellulosic matrix, upon contact with water, the matrix is partially hydrated, forming a gel layer that controls the rate of release of the glutathione. Coating or incorporation of the glutathione granules creates a temporary barrier to dissolution that controls the timing of the delivery of the glutathione in a given environment. Substantial gaps in the matrix allow the glutathione to dissolve quickly, while heavy coating or incorporation slows glutathione dissolution.

Some or all of the ingredients may be screened prior to use using a standard US #20 and/or a standard US #30 mesh screen. The granules may be screened before granulation and again before a milling step. Screening provides granules with a narrower particle or granule size distribution in a range that may be desired for coating and/or compaction and/or the intended use for the composition.

The step of granulating may provide more uniform particles. An active agent can be granulated using any suitable methods known in the art. Granulation is commonly defined as a size-enlargement process in which small particles are gathered into larger, permanent aggregates in which the original particles may still be identified and renders them into a free flowing state. Prior to granulation, a binder may be added to the active agent to improve the granulation process. Other additives may be added during granulation. These include, e.g., sweeteners, flavors, color agents, antioxidants, etc.

Optionally, water or other solvent may be added to aid the granulation process. The amount of water or solvent added depends on, for example, the selection of a granulation process, and is readily determinable by those of skill in the art. Water or other solvent may be added at any suitable time point during the granulation process. For example, a binder may be mixed with a solvent (e.g., water) to form a granulating agent, and then the granulating agent can be sprayed onto active agents. Alternatively, if a granulating agent is too viscous to be uniformly sprayed onto active agents, it may be desirable to blend the binder with the active agent first and then spray water or other solvent to produce a uniform pattern of active agent granules.

A variety of suitable granulation methods can be used to produce granules comprising an active agent. Wet granulation and/or dry granulation methods can for example be used.

Dry granulation refers to the granulation of a composition without the use of heat and solvent. Dry granulation technology often includes slugging or roll compaction. Slugging consists of dry-blending a composition and compressing the composition into a large tablet or slugs on a compressing machine. The resulting tablets or slugs are milled to yield the granules. Roller compaction is similar to slugging, but in roller compaction, a roller compactor is used instead of the tableting machines. See, e.g., Handbook of Pharmaceutical Granulation Technology, D. M. Parikh, eds., Marcel-Dekker, Inc. pages 102-103 (1997). The dry granulation technique is useful in certain instances, for example, when an active agent is sensitive to heat or solvent.

Alternatively, wet granulation can be used. In wet granulation, solvents and binders are typically added to a composition to provide larger aggregates of granules. The temperature during granulation may be set at any suitable point, generally not exceeding the melting point of any components of the composition. The mixture is granulated at a temperature of about 35° C. to about 65° C. for about 20 to about 90 minutes. The mixture may be granulated for less than about 20 minutes, or for about 1 to about 10 minutes at room temperature. Then the granules may be air dried for a suitable duration (e.g., one or more hours).

An active agents may be granulated by high shear mixer granulation (“HSG”) or fluid-bed granulation (“FBG”). Both of these granulation processes provide enlarged granules but differ in the apparatuses used and the mechanism of the process operation. These granulation techniques can be performed using commercially available apparatuses.

In HSG, blending and wet massing are accomplished by high mechanical agitation by an impeller and a chopper. Mixing, densification, and agglomeration of wetted materials are achieved through shearing and compaction forces exerted by the impeller. The primary function of the chopper is to cut lumps into smaller fragments and aid the distribution of the liquid binder. The liquid binder is either poured into a bowl or sprayed onto the powder to achieve a more homogeneous liquid distribution.

Fluidization is the operation by which fine solids are transformed into a fluid-like state through contact with a gas. At certain gas velocities, the fluid will support the particles, giving them freedom of mobility without entrainment. Such a fluidized bed resembles a vigorously boiling fluid, with solid particles undergoing extremely turbulent motion, which increases with gas velocity. Fluidized bed granulation is a process by which granules are produced in a fluidized bed by spraying a granulating agent onto a fluidized powder bed to form larger granules. The granulating agent can be sprayed from, for example, a spray gun positioned in any suitable manner (e.g., top or bottom). The spray position and the rate of spray may depend on the nature of the active agent and the binder used, and are readily determined by those skilled in the art.

Granulating the glutathione may include the steps of premixing the glutathione with a binder such as povidone or Kollidon™ 30 to form a mixture, and granulating the mixture with a granulating agent (granulating vehicle) in a granulator. The granulating agent can be, e.g., povidone or Kollidon™ 30 dissolved in purified water. A high-shear granutator such as a Niro™ PMA 65 High Shear Granulator is often employed. The granulator may be used both to mix the glutathione and binder, and also to granulate the mixture while spraying the granulating vehicle on the mixture.

After the granulation of one or more components of the composition, the granulated composition may be milled. Milling can be performed using any suitable commercially available apparatus (e.g., CoMil equipped with a 0.039 inch screen). The mesh size for the screen can be selected depending on the size of the granules desired. After the granulated active agents are milled, they may be further dried (e.g., in the air) if desired.

Milling the glutathione includes the steps of milling the wet granules or wet milling, drying the granules, and milling the dry granules or dry milling, in accordance with techniques well known in the art (see U.S. Pat. No. 5,145,684 and European Patent Application 498,482). A mill such as a CoMil may be employed to wet mill and dry mill the granules. The mill may be equipped with a '375Q screen for wet milling and a '062R screen for dry milling. The drying step may be accomplished by drying the granules in a bed dryer, e.g., an Aeromatic™ S-2 Fluid Bed Dryer, to a desired Loss on Drying (LOD) level, e.g., a ≦3% LOD. The drying steps can be accomplished in stages until the desired LOD is reached.

Blending the glutathione with the remainder of the ingredients may include a pre-blending step, a blending step, and a final blending step. The pre-blending step may include blending the glutathione/binder granules with a filler and a glidant. e.g., microcrystalline cellulose and colloidal silicon dioxide. The pre-blending step may be accomplished, e.g., in a 0.5 cubic foot V-blender, by blending for about 4 minutes at 25 rpm or in an 8 quart V-Blender, by blending for about 5 minutes at 25 rpm. The blending step may include adding to this blend one or more sustained release agents, e.g., one or more hydroxypropyl methylcelluloses, and a filler, e.g., microcrystalline cellulose. The blending step may be accomplished, e.g., in a 2 cubic foot V-Blender, by blending for about 20 minutes at 25 rpm. The final blending step may include adding a release agent/lubricant, e.g., magnesium stearate, to the blend in the 0.5 cubic foot V-blender and blending for about 4 minutes at 25 rpm.

After preparing the composition as described above, the composition may be compressed into a tablet form. This tablet shaping may be done by any suitable means, with or without compressive force. For example, compression of the composition after the granulation step may be accomplished using any tablet press (e.g., a Manesty™ Beta Press equipped with a 0.748″×0.380″ oval shaped, convex, plain tooling), particularly if the composition is adequately lubricated with lubricant (e.g., magnesium stearate). Many alternative means to affect this step are available, and the invention is not limited by the use of any particular apparatus. The compression step may be carried out using a rotary type tablet press. The rotary type tableting machine has a rotary board with multiple through-holes, or dies, for forming tablets. The composition may be inserted into the die and may be subsequently press-molded.

Alternatively, the tablets may be made by molding. Molded tablets may be made by molding in a suitable machine a mixture of the powdered compound moistened with an inert liquid diluent.

The diameter and shape of the tablet depends on the molds, dies and punches selected for the shaping or compression of the granulation composition. Tablets may be discoid, oval, oblong, round, cylindrical, triangular, and the like. The tablets may be scored to facilitate breaking. The top or lower surface can be embossed or debossed with a symbol or letters.

The compression force may be selected based on the type/model of press, what physical properties are desired for the tablet product (e.g., desired hardness, friability, etc.), the desired tablet appearance and size, and the like. The compression force applied may be such that the compressed tablets have a hardness of at least about 2 kp. Such tablets generally provide sufficient hardness and strength to be packaged, shipped or handled by the user. If desired, a higher compression force may be applied to the tablet to increase the tablet hardness. The compression force is often selected so that it does not deform (e.g., crack or break) the active agent-containing particles within the tablet. The compression force applied may be such that the compressed tablet has a hardness of less than about 10 kp. In certain embodiments, a tablet may be compressed to a hardness of between about 3 kp to about 7 kp, optionally between about 3 kp to about 5 kp, or about 3 kp.

The final tablet may have a weight of about 50 mg to about 2000 mg, or about 200 mg to about 1000 mg, or about 400 mg to about 700 mg.

The compositions and the methods described herein render a composition that achieves a desirable sustained release dissolution profile. A sustained release glutathione composition may sustain in vitro drug release for at least up to 14 hours, at about 10% to about 40% at about 1 hour, about 30% to about 70% at about 4 hours, about 55% to about 75% at about 6 hours, about 65% to about 85% at about 8 hours, about 75% to about 95% at about 12 hours and about 80% to about 100% at 14 hours.

Compositions and methods described herein may render a sustained release glutathione composition that is not excessively friable. Furthermore the composition and methods described herein may render a sustained release glutathione composition that is sufficiently compressible to allow for convenient manufacturing of the composition.

If desired, other modifications may be incorporated into embodiments of the tablet. For example, modification of active agent release through a tablet matrix may also be achieved by any known technique, such as, e.g., application of various coatings, e.g., ion exchange complexes with, e.g., Amberlite™ IRP-69. The tablets of the invention may also include or be coadministered with GI motility-reducing drugs. The active agent may also be modified to generate a prodrug by chemical modification of a biologically active compound that will liberate the active compound in vivo by enzymatic or hydrolytic cleavage, etc. Additional layers or coating may act as diffusional barriers to provide additional means to control rate and timing of drug release.

In another aspect, there is provided a composition for the treatment of various indications, such as treating or preventing Alzheimer's disease or Parkinson's disease, using food as the delivery route. The food may be in the form of a bar such as a prescription health bar. Use of food may enable the provision of larger amounts of glutathione than could be incorporated into a single tablet. A bar that may provide more than 1 gram of glutathione, as well as other agents as desired, is provided. In one embodiment, the glutathione may be added as an immediate release composition, e.g., immediate release granules of glutathione, to a food bar. The bar may include a sustained release composition that includes, for example, sustained release granules of glutathione. The granules may include taste masking constituents, e.g., taste making coatings. Use of food may also reduce the need for taking multiple tablets of glutathione when a higher dose is desired.

The food bars may have between about 1 and about 10 grams of glutathione. The bars may be provide having a total of about 4 g per bar of glutathione or its salts in conjunction with sugars, fruit components, protein, and vitamins and minerals. An individual bar may weigh in the range of about 25 g to about 100 g. In a particular process, the bar may be produced by combining sugars and fruit paste at an elevated temperature and then combining the syrup at a reduced temperature with the minor ingredients. After blending the minor ingredients in the syrup, the glutathione may be added. The glutathione may be added in conjunction with a protein extender, followed by bulking and food agents, e.g. fruit pieces or other particulate edible ingredients providing the desired texture and flavor, and soy proteins. Methods and compositions for manufacturing health bars with glutathione and L-lysine are described in, e.g. U.S. Pat. No. 6,063,432.

In another aspect, there is provided a method of manufacturing a food bar as described herein. The method may include granulating the glutathione, as described herein. The granulating step may include a pre-mixing step and a granulating step. The method may also include a wet milling step, as described herein. Such a bar may be obtained by wet granulation of the glutathione with appropriate excipients, such as described herein. The resulting granules may be used as is or coated with a taste masking cellulosic.

This invention is further illustrated by the following examples that should not be construed as limiting.

### EXAMPLE 1

A composition comprising 70% Glutathione, 2.8% Kollidon™ 30, 21.7% Methocel™ KM100P CR, 5.5% Methocel™ E4M CR. was prepared using the following method.

5 kilograms of Glutathione and 50 grams of Kollidon™ 30 were mixed for 4 minutes in a Bohle™ VMA20. A solution of 150 grams Kollidon™ 30 was prepared using 0.633 kilograms of purified water. Using a small nozzle the Kollidon™ 30 solution was added over a roll time duration of 6.5 minutes. The granulate was dried using a fluidbed granulator. Granulation was sized to 100% minus 20 U.S. std. mesh. 1.55 kilograms of Methocel™ K100M CR and 393 grams Methocel™ E4M CR were added to the above granulation in a 0.5 cubic foot (or larger, if necessary) V-blender through a 20 U.S. std. mesh screen. The materials were blended for four minutes and then dropped through a 20 U.S. std. mesh screen. The blend was re-introduced into the 0.5 cubic foot (or larger, if necessary) V-blender and blended for another four minutes. The blend was then discharged into a plastic pale with desiccants.

The resulting 70% Glutathione, 2.8% Kollidon™ 30, 21.7% Methocel™ KM100P CR, 5.5% Methocel™ E4M CR preparation was then mixed with a small amount of silicon dioxide and Leucine and capsuled.

### EXAMPLE 2

A sustained release glutathione formulation of the invention, formulated in accordance with Example 1, was administered to patient YL. Patient YL had been diagnosed as suffering from Alzheimer's disease. and exhibited characteristic symptoms of associated severe memory deficit. Patient YL responded to oral administration of the sustained release formulation with a dramatic improvement in memory. Treatment of the patient with glutathione was temporarily suspended. During suspension of treatment, a rapid relapse of memory deficit occurred. Treatment with sustained release glutathoine was resumed, and patient YL, subsequently exhibited a return to an improved memory state.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

1. A composition suitable for oral administration for sustained-release of glutathione, the composition comprising:
a) from about 50% by weight to about 90% by weight glutathione;
b) from about 0% by weight to about 10% by weight binder;
c) from about 10% by weight to about 50% by weight of at least one sustained release agent; and
d) a granule size of from about 500 μm to about 5000 μm.

2. The composition of claim 1 wherein said composition provides sustained release of glutathione over a period of from about 2 hours to about 12 hours.

3. The composition of claim 1 wherein said composition provides sustained release of glutathione over a period of from about 4 hours to about 8 hours.

4. The composition of claim 1 wherein the granule size is from about 500 μm to about 2000 μm.

5. (canceled)

6. (canceled)

7. (canceled)

8. (canceled)

9. The composition of claim 1 further comprising a glidant.

10. The composition of claim 9 wherein the glidant is silicon dioxide.

11. The composition of claim 1 further comprising leucine.

12. The composition of claim 1 wherein the glutathione is present at from about 60% by weight to about 80% by weight.

13. (canceled)

14. (canceled)

15. The composition of claim 1 wherein the binder is present at from about 0% by weight to about 5% by weight.

16. (canceled)

17. The composition of claim 16 wherein the binder is Kollidon™ 30.

18. (canceled)

19. The composition of claim 1 wherein the at least one sustained release agent is present at from about 20% by weight to about 40% by weight.

20. (canceled)

21. (canceled)

22. The composition of claim 1 wherein the at least one sustained release agent is selected from at least one of the group consisting of: Methocel™ KMlOOP CR, and Methocel™ E4M CR.

23. (canceled)

24. (canceled)

25. The composition of claim 1 where the at least one sustained release agent comprises Methocel™ KMl 00P CR and Methocel™ E4M CR.

26. The composition of claim 25 wherein the Methocel™ KMl 00P CR is present at about 21.7% by weight and the Methocel™ E4M CR is present at about 5.5% by weight.

27. The composition of claim 1 suitable for administration to a patient having a gastric pH of from about 1 to about 7.

28.-41. (canceled)

42. A composition comprising about 70% by weight glutathione, about 2.8% by weight Kollidon™ 30, about 21.7% by weight Methocel™ KMlOOP CR, about 5.5% by weight Methocel™ E4M CR and having a granule size not more than about 850 μm.

43. A composition comprising about 72% by weight Glutathione, about 22.3% by weight Methocel™ KMlOOP CR, about 5.7% by weight Methocel™ E4M CR and having a granule size not more than about 850 μm.

44. The composition of claim 42 or 43 wherein the granule size is about 850 μm.

45. A composition containing about 70% by weight glutathione, about 2.8% by weight Kollidon™ 30, about 21.7% by weight Methocel™ KMlOOP CR, about 5.5% by weight Methocel™ E4M CR having a granule size not more than about 850 μm

46. A composition containing about 72% by weight Glutathione, about 22.3% by weight Methocel™ KMlOOP CR, about 5.7% by weight Methocel™ E4M CR and having a granule size not more than about 850 μm.

47. The composition of claim 45 or 46 further containing a trace amount of leucine and a trace amount of silicon dioxide.

48. A method for preparing a sustained-release composition comprising glutathione, suitable for oral administration, the method comprising:
mixing glutathione and a binder, thereby forming a mixture;
adding a granulating agent to the mixture, thereby forming a wet mixture;
granulating the wet mixture, thereby forming a granulation, such that the granulation comprises granules larger than about 850 μm;
removing granules less than about 500 μm from the granulation;
blending at least one sustained release agent with the granulation, thereby forming a blend;
screening the blend with an 850 μm mesh, thereby forming a screened blend; and
reblending the screened blend.

49. The method of claim 48 wherein the mixing occurs for a duration of from about 2 minutes to about 6 minutes.

50. (canceled)

51. The method of claim 48 wherein the adding occurs for a duration of from about 4 minutes to about 9 minutes.

52. The method of claim 48 wherein the adding occurs for a duration of about 6.5 minutes.

53. A method for preparing a sustained-release composition comprising glutathione, suitable for oral administration, the method comprising:
blending two sustained release agents together, thereby forming a blend;
screening the blend with a 20 US standard mesh screen, thereby forming a screened-blend;
granulating the screened-blend thereby forming a granulation;
reblending the granulation with glutathione thereby forming a re-blend;
rescreening the re-blend with a 20 US standard mesh screen thereby forming a re-screened-blend;
regranulating the re-screened-blend thereby forming a re-granulation; and
rescreening the re-granulation with a 20 US standard mesh screen.

54. The method of claims 48 or 53 further comprising mixing a glidant with said composition.

55. The method of claims 48 or 53 further comprising mixing leucine with said composition.

56. The method of claims 48 or 53 further comprising encapsulating said composition.

57. The method of claims 48 or 53 wherein the blending occurs for a duration of from about 2 minutes to about 6 minutes.

58. (canceled)

59. The method of claims 48 or 53 wherein the reblending occurs for a duration of from about 2 minutes to about 10 minutes.

60.-61. (canceled)

62. A method of medical treatment comprising orally administering a composition for sustained release of glutathione to a patient suffering from or at risk of suffering from a neurological disorder.

63. The method of medical treatment of claim 62 wherein the neurological disorder is selected from the group consisting of: Multiple Sclerosis, Autism, Alzheimer's disease, Parkinson's disease, Aphasia, Bell's Palsy, Creutzfeldt-Jakob disease, Epilepsy, Encephalitis, Huntington's disease, neuromuscular disorders, neuro-oncology, neuro-immunology, dementia and neuro-otology.

64.-67. (canceled)

68. A method of medical treatment comprising administering the composition of claim 1 to a patient in need thereof, thereby maintaining a serum glutathione concentration in a patient for a duration of from about 2 hours to about 12 hours for the treatment of a neurological disorder.

69. A method of medical treatment comprising administering the composition of claim 1 to a patient suffering from or at risk of suffering from a neurological disorder.

70. A method of medical treatment comprising administering the composition of claim 1 to a patient suffering from or at risk of suffering from at least one of the neurological disorders selected from the group consisting of: Multiple Sclerosis, Autism, Alzheimer's disease, Parkinson's disease, Aphasia, Bell's Palsy, Creutzfeldt-Jakob disease, Epilepsy, Encephalitis, Huntington's disease, neuromuscular disorders, neuro-oncology, neuro-immunology, dementia and neuro-otology.

71.-75. (canceled)

76. The method of any one of claims 68 to 70 wherein the patient is further suffering from low gastric acidity.

77.-118. (canceled)
